(19) United States
(12) Patent Application Publication
(10) Pub. No.: US 2011/0257548 A1
(43) Pub. Date: Oct. 20, 2011

(54) METHOD AND SYSTEM FOR PROCESSING HEART SOUND SIGNALS

(75) Inventors: Liang Dong, Shanghai (CN); Zhongtao Mei, Shanghai (CN); Runze Wu, Shanghai (CN); Maarten Leonardus Christian Brand, Shanghai (CN)

(73) Assignee: KONINKLIJKE PHILIPS ELECTRONICS N.V., EINDHOVEN (NL)

(21) Appl. No.: 13/141,771

(22) PCT Filed: Dec. 22, 2009
(86) PCT No.: PCT/IB09/55896

§ 371 Date: Jun. 23, 2011

(30) Foreign Application Priority Data
Dec. 30, 2008 (CN) 200810190251.5

(51) Int. Cl. A61B 5/02

(52) U.S. Cl. 600/528

(57) ABSTRACT

The invention provides a method of processing at least one heart sound signal, and the method comprises the step of: receiving (11) the at least one heart sound signal, segmenting (12) the heart sound signal into a plurality of segments, identifying (13) attribute information for each segment, annotating (14) each segment with corresponding attribute information, and outputting (15) an annotated Phonocardiogram for the at least one heart sound signal. The invention also provides a processing system for implementing the step of the methods as mentioned above.

### FIELD OF THE INVENTION

The invention relates to a method and system for processing sound signals, particularly, relates to a method and system for processing heart sound signals.

### BACKGROUND OF THE INVENTION

Based on different heart sound sources, a heart sound signal detected from a stethoscope may comprise different type of segments, e.g. S1 segment caused by the closing of mitral and tricuspid valves, S2 segment caused by the closure of aortic and pulmonary valves, S3 segment caused by fast ventricular filling during early diastole, S4 segment caused by atrial contractions displacing blood into the distended ventricular, murmurs may be caused by turbulent blood flow. Sometimes, different type of segment may reflect different specific abnormal heart sound. Furthermore, a heart sound signal may also comprise a plurality of heart cycles (heart beat), and some abnormal heart sound can only be reflected by some specific heart cycles.

Listening to a heart sound from a traditional stethoscope, it may be possible for people to make a general diagnosis depending on his/her experience. However, it is very difficult for people to accurately make a diagnosis for an abnormal heart sound caused by some specific heart sound sources or heart sound cycles because of the limitation of human ears, even he/she is every experienced in auscultation area.

In the past years, many technologies have been developed for digital stethoscope to output accurate and reliable PCG (Phonocardiogram), so that people can make a diagnosis based on PCG easily, instead of listening. A PCG outputted by a current digital stethoscope is an almost raw PCG. Based on a raw PCG, people still have to identify an abnormal heart sound caused by some specific heart sound source or some specific heart cycles by his/her experience mostly.

Thus, the current digital stethoscope cannot give very intelligent indication for helping people to make a diagnosis accurately and conveniently.

### SUMMARY OF THE INVENTION

An object of this invention is to provide a method for processing at least one heart sound signal, so as to output at least one more understandable Phonocardiogram.

The invention provides a method of processing at least one heart sound signal, and the method comprises the step of:
receiving the at least one heart sound signal,
segmenting the heart sound signal into a plurality of segments,
identifying attribute information for each segment,
annotating each segment with corresponding attribute information, and
outputting an annotated Phonocardiogram for the at least one heart sound signal.

The advantage is that the annotated Phonocardiogram is more understandable, so that people can make a diagnosis more accurately and conveniently.

In another embodiment of the invention, the method also comprises a step of comparing two annotated Phonocardiograms to acquire a comparison result, if the at least one heart sound signal comprises multiple heart sound signals, and the multiple heart sound signals come from different heart sound sources respectively, wherein,

the annotating step is further intended to annotate the comparison result on any one of the Phonocardiograms which are compared with each other to form a comparison Phonocardiogram, and

the outputting step is further intended to output the comparison Phonocardiogram.

The advantage is that, based on the comparison PCG, two annotated PCGs complement with each other to provide more accurate information for people to make a diagnosis.

In a further embodiment of the invention, the method also comprises a step of generating a heart rate information table for the at least one heart sound signal by extracting heart cycle samples from the heart sound signal, and the heart rate information table comprises different heart rate categories, a typical heart cycle Phonocardiogram for each heart rate category, and an annotated heart cycle Phonocardiogram for each heart rate. The outputting step is further intended to output the heart rate information table for the heart sound signal.

The advantage is that, based on the heart rate information table, people can easily identify abnormal heart sounds and further learn at which heart rate the heart condition of the patient becomes worse.

The invention also provides a processing system for implementing the steps of the method as mentioned above.

Detailed explanations and other aspects of the invention will be given below.

### DESCRIPTION OF THE DRAWINGS

The above and other objects and features of the present invention will become more apparent from the following detailed description considered in connection with the accompanying drawings, in which:

FIG. 1 is a schematic diagram for illustrating an embodiment of the method according to the invention;

FIG. 2 is a graph for illustrating a raw Phonocardiogram for a heart sound signal;

FIG. 3 is a graph for illustrating multiple raw Phonocardiograms for multiple heart sound signals;

FIG. 4 is a graph of illustrating a segmented heart sound signal;

FIG. 5 is a statistical histogram illustrating an appearance frequency of each interval range of segments;

FIG. 6 is a graph illustrating the relationship between an Electrocardiogram and a corresponding synchronized Phonocardiogram;

FIG. 7 depicts two annotated Phonocardiograms;

FIG. 8 depicts a schematic arrangement for multiple sensors to detect multiple heart sound signals;

FIG. 9 depicts a comparison Phonocardiogram for aortic area Phonocardiogram and tricuspid Phonocardiogram;

FIG. 10 is a schematic graph for illustrating extracting heart cycle samples from a heart sound signal based on R wave;

FIG. 11 depicts a heart rate information table;

FIG. 12 is a schematic diagram for illustrating a stethoscope;

FIG. 13 depicts a processing system for processing at least one heart sound signal in accordance with an embodiment of the stethoscope of FIG. 12.

The same reference numerals are used to denote similar parts throughout the figures.

### DETAILED DESCRIPTION

The method of the invention is to process at least one heart sound signal for outputting a more understandable Phonocardiogram (called PCG in the following), so that people can make a diagnosis conveniently and accurately.

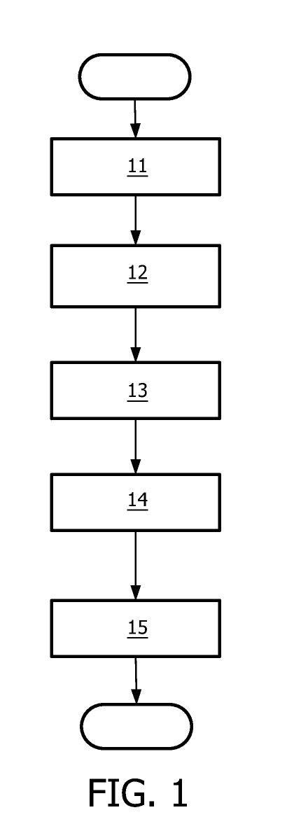

FIG. 1 is a schematic diagram for illustrating one embodiment of the method according to the invention. The method for processing at least one heart sound signal comprises the following steps:
receiving 11 at least one heart sound signal;
segmenting 12 the at least one heart sound signal into a plurality of segments;
identifying 13 attribute information of each segment;
annotating 14 each segment with corresponding attribute information; and
outputting 15 an annotated PCG for the segments.

(1) Receiving 11 at Least One Heart Sound Signal

The at least one heart sound signal may comprise one heart sound signal, or multiple heart sound signals coming from different heart sound sources. The multiple heart sound signals can be two or more heart sound signals. Each heart sound signal is detected by sound sensor placed on a heart sound source, such as mitral area, tricuspid area, aortic area, pulmonary area.

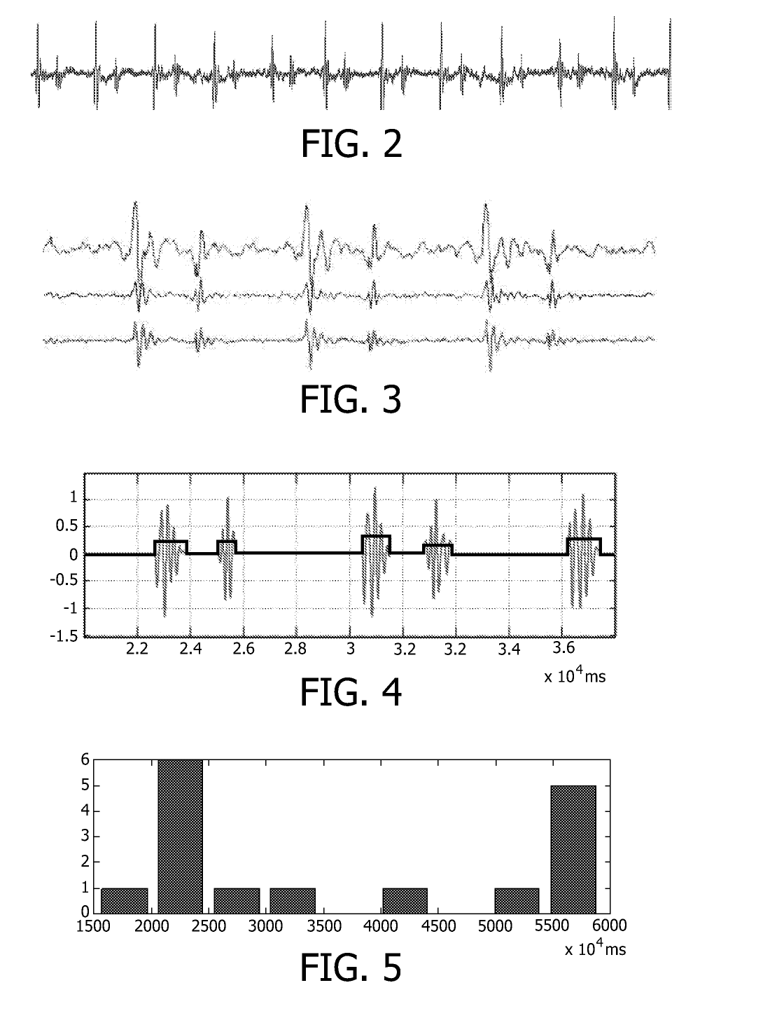

FIG. 2 is a graph for illustrating a raw PCG for a heart sound signal, and FIG. 3 is a graph for illustrating multiple raw PCGs for multiple heart sound signals.

A heart sound signal may comprise several segments which belong to different signal segment types, for example, S1 segment, S2 segment, S3 segment, S4 segment, murmurs segment. S1 is caused by the closure of mitral and tricuspid valves; S2 occurs during the closure of aortic and pulmonary valves; S3 is due to the fast ventricular filling during early diastole; S4 occurs as the result of atria contractions displacing blood into the distended ventricular; murmurs are most likely to be caused by turbulent blood flow. S1 may further comprise M1 caused by Mitral and T1 caused by tricuspid, and S2 may further comprise A2 caused by Aortic and P2 caused by Pulmonic valves. For healthy individuals, S3, S4 and murmurs are usually inaudible.

(2) Segmenting 12 at Least One Heart Sound Signal into a Plurality of Segments

If the at least one sound signal comprises multiple heart sound signals, the segmenting step 12 is used to segment the multiple heart sound signals separately.

The first embodiment of the segmenting step 12 may comprise the steps of:
filtering the heart sound signal by a band-pass filter for selecting a wave band of the heart sound signal, wherein the wave band is a predefined frequency range. The filtering step is intended to cut-off frequency 10-100 Hz from the heart sound signal for selecting the wave band within the predefined frequency range. The predefined frequency range is predefined according to the energy of a heart sound signal, since some segments of a heart sound signal have very prominent energy corresponding to a specific frequency range. After filtering the heart sound signal, some high frequency noise (such as lung sounds) and some low frequency noise (such as baseline drift) can be eliminated.
extracting segments from the wave band, if the average amplitude change rate of a segment is higher than a predefined change rate threshold. For example, the 5-10% segments, which have average amplitude change rates being higher than the predefined change rate threshold, are extracted from the wave band. Normally, the segments of a heart sound wave, such as S1, S2, S3, S4, murmurs, are corresponding to peaks/valleys where the amplitude change is more intensive than the baseline part. The extracting step may be further intended to merge adjacent blocks, and then smooth the edges of each segment.

The second embodiment of the segmenting step is intended to segment a heart sound signal based on evelogram. Based on the second embodiment, the segmenting step may comprise:
filtering the heart sound signal into an envelogram. The filtering step can be implemented by Hilbert transform, Homomorphic transform, or curve fitting transform. Curve fitting transform: in a heart sound signal waveform, the outlier points, e.g. maximum points, can be detected easily, so Quadric curves, which may be B-splines, parabolas or Beziers, can then be used to connect these points to build the envelogram.
extracting segments from the envelogram, if the average amplitude of a region around a peak point of the heart sound signal exceeds a predefined amplitude threshold. The extracting step may be further intended to merge adjacent blocks, and then smooth the edges of each segment.

FIG. 4 is a graph of illustrating the segmented heart sound signal according to the first embodiment and the second embodiment of the segmenting step. The X-coordinate represents time, and the Y-coordinate represents amplitude.

(3) Identifying 13 Attribute Information of Each Segment

The attribute information comprises the type of each segment, the duration of each segment, the timing of each segment, the amplitude of each segment, and/or the intensity of each segment etc. The type of each segment can be S1, S2, S3, S4, and murmurs.

The identifying step 13 may be intended to identify the attribute information of each segment according to the waveform of each segment, relationships of the segments, or jointing an Electrocardiogram (called ECG in the following) with the PCG of the heart sound signal, wherein the signal of the ECG is synchronous with the heart sound signal. Four examples are given in the following for explaining the identifying step 13.

The first embodiment for the identifying step is based on the relationship of the segments. In this embodiment, the identifying step may comprise:
determining intervals between peak points of the segments to form a statistical histogram, wherein the intervals is divided into different interval ranges, and the statistical histogram reflects the appearance frequency of each interval range.

FIG. 5 is a statistical histogram illustrating an appearance frequency of each interval range of segments.
determining the interval range between S1 segment and S2 segment (in the following, called interval S1-S2) in the statistical histogram, wherein the appearance frequency of the interval S1-S2 is the highest in the statistical histogram. The interval S1-S2 is stable within a short period, e.g. 10 seconds, so in the statistical histogram, the interval S1-S2 usually appears most frequently. In FIG. 5, the interval within 2000˜2500 sample units (or 0.25˜0.31 second at the sampling rate of 8 KHz) appears 6 times which is the highest appearance frequency and can determined as the interval S1-S2. In FIG. 5, the X-coordinate represents time, and the Y-coordinate represents amplitude.
determining the interval range between S2 segment and S1 segment (in the following, called interval S2-S1) in the statistical histogram, wherein the appearance frequency of the interval S2-S1 is only less than the appearance of the interval S1-S2. Similarly, the interval S2-S1 is also stable within a short period and is longer than interval S1-S2. In FIG. 5, the interval within 5500˜6000 sample units (or 0.69˜0.75 second at the sampling rate of 8 KHz) appears 5 times, which is only less the appearance frequency of S1-S2 interval, and then can be determined as the interval S2-S1.
determining S1 segment and S2 segment based on the interval S1-S2 and interval S2-S1. The S1 and S2 segments are identified by entirely searching the wave of the heart sound signal based on the S1-S2 interval and S2-S1 interval. For example, if the interval between any two consecutive peaks is within the S1-S2 interval as shown in FIG. 5, e.g. 2000˜2500 sample units, the segment corresponding to the previous peak is determined as S1, and the subsequent peak corresponds to S2.
determining S3 segment, S4 segment, and murmur based on the determined S1 and S2 in the same heart sound cycle and the position information of S3 segment, S4 segment, and murmur.
determining the split of S1 segment and S2 segment by performing homonorphic filtering and peak detection to identify M1 segment, T1 segment, A2 segment, and P2 segment.
determining duration, amplitude, timing, and intensity for each segment.

The second embodiment for the identifying step 13 is based on the waveform of each segment. The identifying step may comprise the steps of:
determining S1 segment and S2 segment by detecting peaks along the segments, wherein S1 segment and S2 segment are corresponding to the first highest and the second highest peaks in the envelogram respectively. The envelogram is formed during the segmenting step 12 (the second embodiment of the segmenting step).
determining S3 segment, S4 segment, and murmur based on the determined S1 and S2 in the same heart sound cycle and the positions of S3 segment, S4 segment, and murmur.
determining the split of S1 and S2 by performing homonorphic filtering and peak detection.
determining duration, amplitude, timing and intensity for each extracted segment according to the waveform of each segment.

The third embodiment for the identifying step 13 is based on the waveform of each segment. In this embodiment, the identifying step 13 may comprise:
detecting the heart sound cycles of the at least one heart sound signal.
determining the type of each segment in the heart sound signal by the way of Hidden Markov Model (HMM), or Neural Network, or Linear/Dynamic Time Warping. The type of segment can be S1 segment, S2 segment, S3 segment, S4 segment, murmur etc.
determining the split of S1 segment and S2 segment by performing homonorphic filtering and peak detection to identify M1 segment, T1 segment, A2 segment, and P2 segment.
determining duration, amplitude, timing and intensive for each extracted segment according to the waveform of each segment.

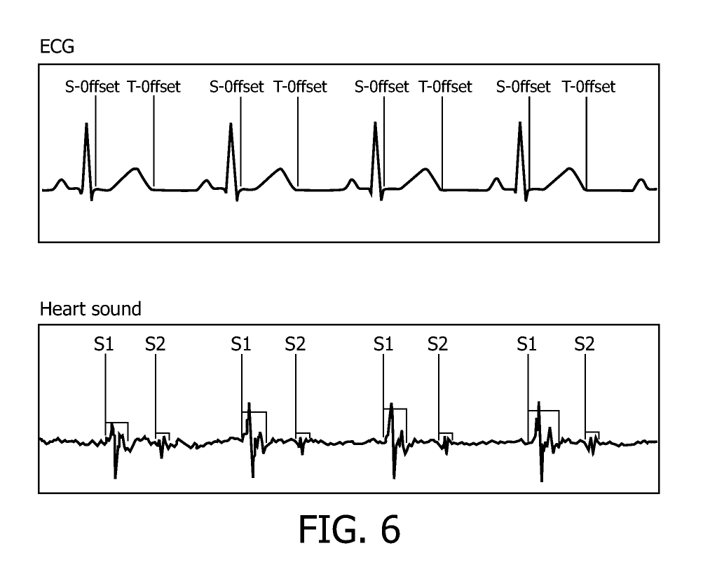

The fourth embodiment for the identifying step 13 is based on jointing ECG and corresponding synchronized PCG. In this embodiment, the identifying step 13 may comprise:
receiving an ECG, wherein the at least one heart sound signal and the signal of ECG are synchronous.
detecting key points of the ECG, wherein the key points comprise S-onset, S-offset, T-onset, T-offset, wherein the S-offset of the ECG indicates the beginning of S1 segment and the T-offset corresponds to the beginning of S2 segment in the time domain.
Mapping the key points of the ECG to the segments of the PCG to determine the type of each segment. The S-offset and T-offset can be detected on the ECG signal with many approaches such as Wavelet transform, Hidden Markov Model, etc. And based on the relationship between ECG and PCG, the starting points of S1 and S2 can be determined FIG. 6 is a graph illustrating the relationship between an ECG and a corresponding synchronized PCG.
determining S3 segment, S4 segment, and murmur based on the determined S1 and S2 in the same heart sound cycle and the position information of S3 segment, S4 segment, and murmur.
determining the split of S1 segment and S2 segment by performing homonorphic filtering and peak detection to identify M1 segment, T1 segment, A2 segment, and P2 segment.
determining duration, amplitude, timing and intensive for each extracted segment according to the waveform of each segment.

(4) Annotating 14 Each Segment with Corresponding Attribute Information

The annotating step 14 is intended to annotate each segment with the type of S1, S2, S3, S4, or murmur according to the identified attribute information. The annotating step 14 is further intended to annotate each segment with amplitude, duration, intensity etc. according the identified attribute information.

(5) Outputting 15 an Annotated PCG for the Heart Sound Signal

The outputted PCG comprises a plurality of segments, and each segment is annotated with corresponding type, amplitude, duration, intensity, timing etc., so that people can recognize problems of the heart sound signal conveniently and accurately.

The annotated Phonocardiogram is to be displayed in the form of bar-shaped diagram, and the height of a bar indicates the average amplitude of each segment, and the width of a bar indicates the duration of each segment.

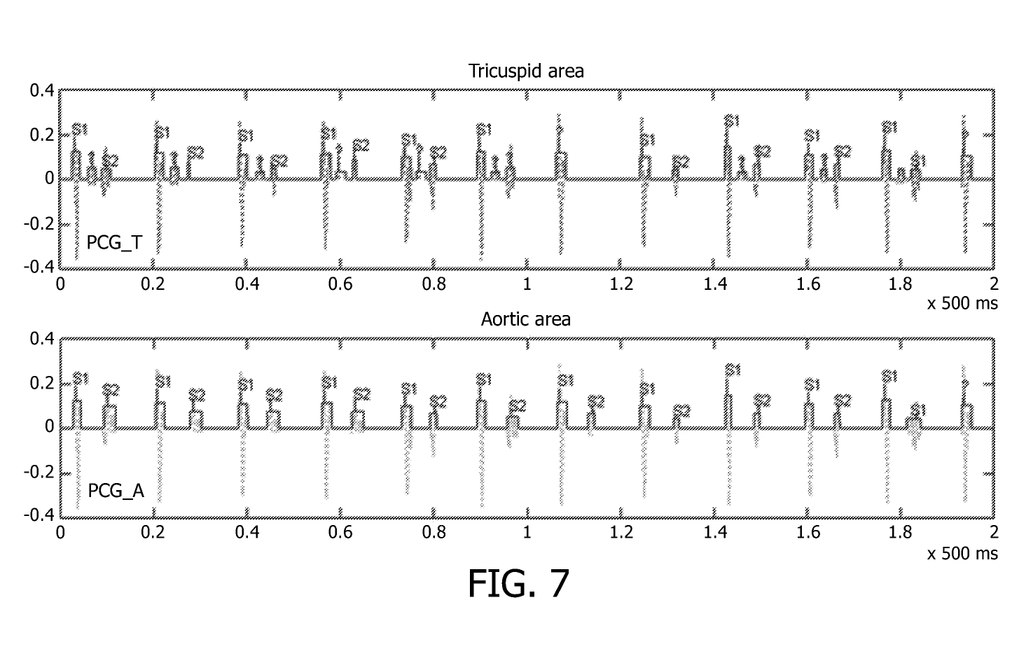

FIG. 7 depicts two annotated PCGs, the non-recurrent segments, which are treated as noise, are indicated as “?”. In FIG. 7, the two annotated PCGs come from the heart sound source of aortic (S2) area and tricuspid (S1) area, so S3 segment and S4 segment are not prominent to be shown.

The method of processing at least one heart sound signal further comprises a comparing step and a generating step (not shown in FIG. 1).

(6) Comparing Step

Comparing two annotated PCGs to acquire a comparison result, if the at least one heart sound signal comprises multiple heart sound signals, and the multiple heart sound signals come from different heart sound sources respectively. The comparison result comprises similarities and differences of any two annotated PCGs which are compared with each other.

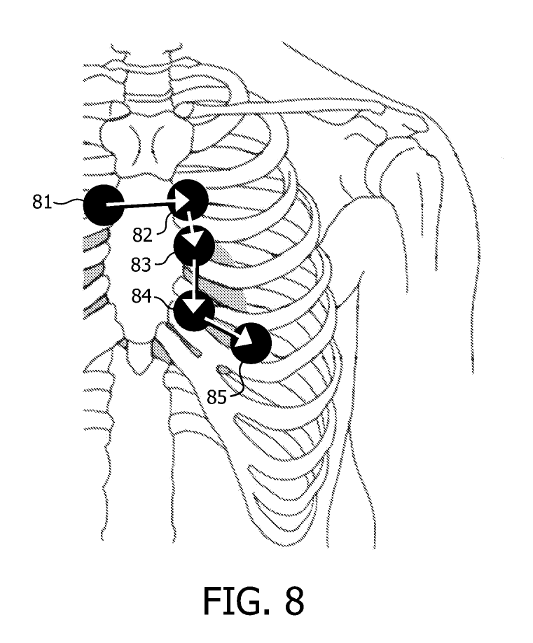

FIG. 8 depicts a schematic arrangement for multiple sensors to detect multiple heart sound signals. The arrangement comprises five combined sensors, and every combined sensor may comprise a PCG sensor and an ECG sensor. The five combined sensors are placed on aortic area 81, pulmolic area 82, erb's point 83, tricuspid area 83, and mitral area 85 respectively for detecting heart sound signals.

The annotating step 14 is further intended to annotate the comparison result on any one of the PCGs which are compared with each other to form a comparison PCG.

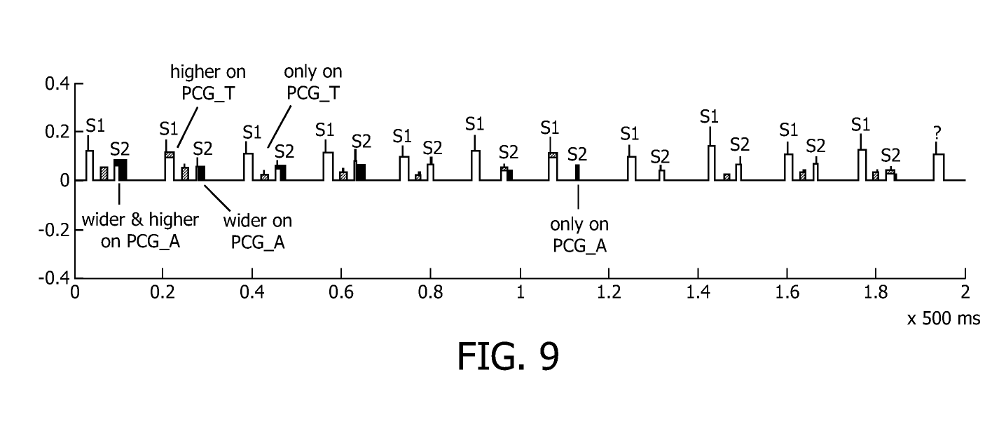

The outputting step 15 is further intended to output the comparison PCG. FIG. 9 depicts a comparison PCG for aortic area PCG and tricuspid PCG, the X-coordinate represents time, and the Y-coordinate represents amplitude.

The comparing step is intended to compare the average amplitude and the duration of two annotated PCGs. For example, one annotated PCG is from tricuspid area (denoted as PCG_T in the following) and another annotated PCG is from aortic area (denoted as PCG_A in the following). In PCG_A, S2 has bigger amplitude and longer duration, so S2 of PCG_A is more easily identified, then the annotating step 14 is intended to annotate “wider & higher on PCG_A” for this S2 segment on the comparison PCG. In some cases, S2 is not detected on PCG_T, but it can be correctly identified on PCG_A, and then the annotating step 14 is intended to annotate on the comparison PCG “only on PCG_A” for this S2 segment. The comparison PCG can be generated based on PCG_A or PCG_T.

Based on the comparison PCG, two PCGs complement with each other to provide more accurate information than using single-channel PCG. Furthermore, the presence of abnormal heart sounds, e.g. S3, S4 and murmurs, can be determined conveniently based on the comparison PCG.

Some recurrent sounds are detected on PCG_T but not on PCG_A, and the segments of the recurrent sounds are annotated as “only on PCG_T”, which shows that the recurrent sounds are not noise, and the source of the sound is near tricuspid area but far from aortic area. Furthermore, several kinds of murmurs appear between S1 segment and S2 segment, such as systolic ejection murmurs, ventricular outflow obstruction murmurs, systolic regurgitation murmurs, ventricular septal defect murmur. The comparison PCG reflects the ventricular septal defect murmur very well because such murmur sound is easily audible at PCG_T but not distinct at the PCG_A. In this way, a physician can reach fast and accurate conclusion to the heart condition.

(7) Generating Step

Generating a heart rate information table for the heart sound signal by extracting heart cycle samples from the heart sound signal, wherein the heart rate information table comprises different heart rate categories, a typical heart cycle PCG for each heart rate category, and an annotated heart cycle PCG for each heart rate which is formed by steps 12-14 as shown in FIG. 1.

The outputting step 15 is also intended to output the heart rate information table for the heart sound signal.

The heart cycle samples are extracted by jointing an ECG and the PCG of the heart sound signal which is synchronous with the ECG signal.

The generating step comprises:
receiving an ECG signal, wherein the ECG signal and the heart sound signal are synchronous.
extracting heart cycle samples from the heart sound signal by making use of the periodicity of the appearances of R waves and R-peak as a beat delimiter for both ECG and the PCG of the heart sound signal, wherein the R wave is the steepest wave along the ECG waveform and the R-peak is the peak point of the R wave.

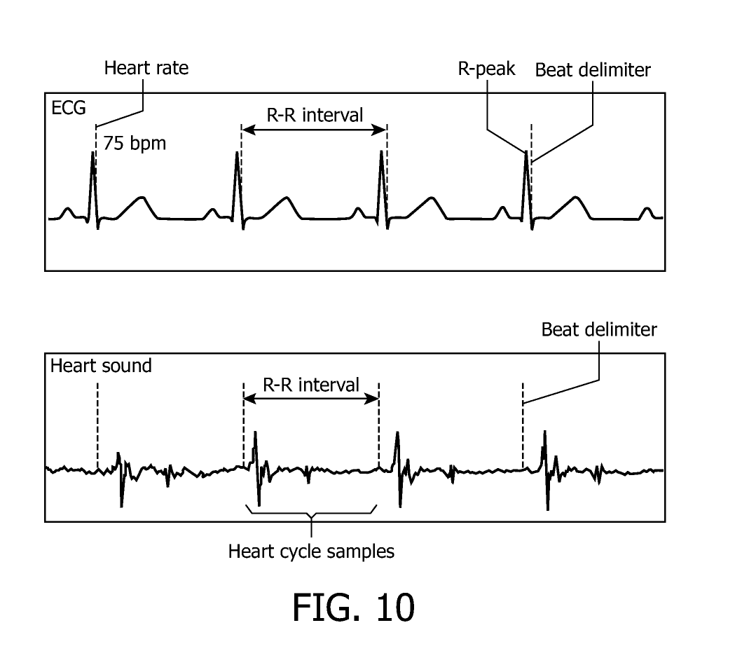

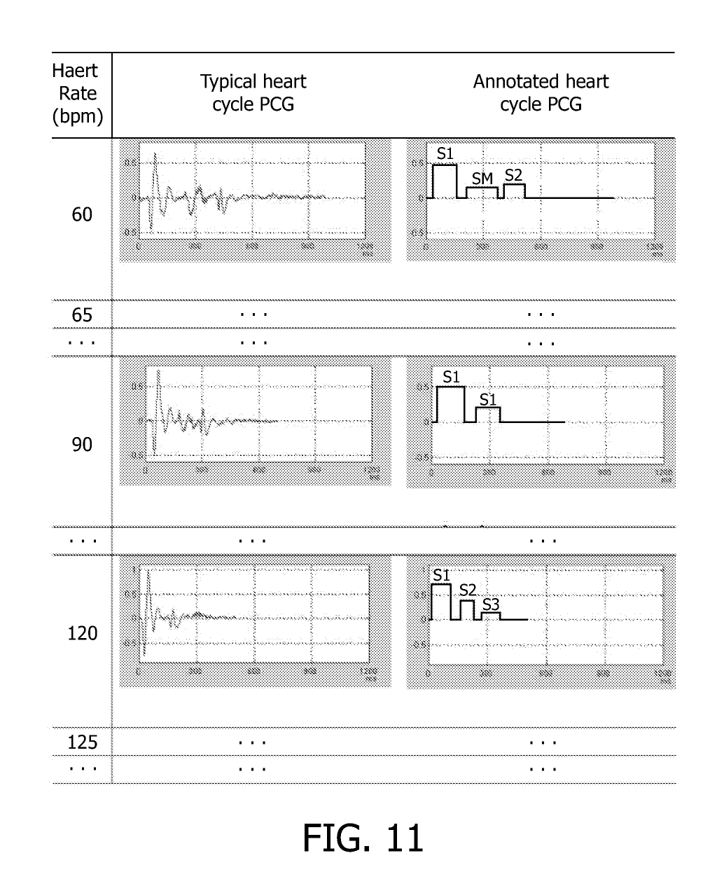

FIG. 10 is a schematic graph for illustrating extracting heart cycle samples from a heart sound signal. The ECG region of two consecutive R-peaks, namely R-R interval, is a heart beat, and the region in an R-R interval is referred as a heart cycle sample.
Calculating heart rate for each heart cycle sample. For example, if the heart cycle is 1 second, then the heart rate corresponding to the heart cycle is 60 beats/minute.
Categorizing the heart cycle samples into different heart rate categories, wherein the heart cycles in the same heart rate category have the same heart rate.
eliminating noise by adding all heart cycle samples of the same heart rate together to forming a typical heart cycle PCG for the heart rate. For example, to directly add the aligned bit of amplitude values of the heart cycle samples to eliminate noise. The heart cycle samples include S1, S2, S3, S4, murmurs (if there are murmurs) which are recurrent and demonstrate strong similarity between one heart cycle and another. The eliminating step will not affect the quality of the heart cycle samples. The noise, on the other hand, is Gaussian-like, and can be counteracted by accumulation operation. The new data sequence generated by adding the heart cycle samples is referred as typical heart cycle, which has higher SNR (Signal-Noise Rate) than the heart cycle samples. And the more heart cycle samples are accumulated, the higher SNR is achieved. For example, if 20 heart cycle samples are summed up for the same heart rate category, the SNR increases approximately 20 dB. It should note that for the same heart rate, the length of heart cycle samples is almost identical. Thus the heart cycle samples can be added up without or with minor truncation/stretching.
forming a heart rate information table, wherein the heart rate information table comprises different heart rate categories, a typical heart cycle PCG for each heart rate category, and an annotated heart cycle PCG for each heart rate category. FIG. 11 depicts a heart rate information table, for typical heart cycle PCG and annotated heart cycle PCG, Y-coordinate represents amplitude, and X-coordinate represents time.

Based on heart rate information table, some murmurs, e.g. systolic murmur (SM) in this instance, can be observed at lower heart rate, say 60 bpm (60 beats/minute), where the interval between S1 and S2 is longer, and the intensity of S1 and S2 are lower. At higher heart rate, e.g. 90 bpm and above, systolic murmur is swarmed by S1 and S2, because S1-S2 interval becomes shorter and their average intensities are higher. Other abnormal heart sound, e.g. S3, is weak at low heart rate but got enhanced as heart rate increases (e.g. 120 bpm), and can be detected on the typical heart cycle PCG and annotated heart cycle PCG. This is due to the fact that S3 is associated with blood volume and velocity. The higher the heart rate, the faster the velocity of blood flow, and in turn produces more easily detectable S3 on the typical heart cycle PCG and the annotated heart cycle PCG.

When presented with such a heart rate information table, people may easily identify abnormal heart sounds and further learn at which heart rate the heart condition of the patient becomes worse.

The heart sounds at different auscultation areas (heart sound sources) on the chest can be acquired using multiple heart sound sensors and processed in the same manner. The heart rate information table may comprise heart sound information for multiple auscultation areas, which can be more informative to people than for only one auscultation area.

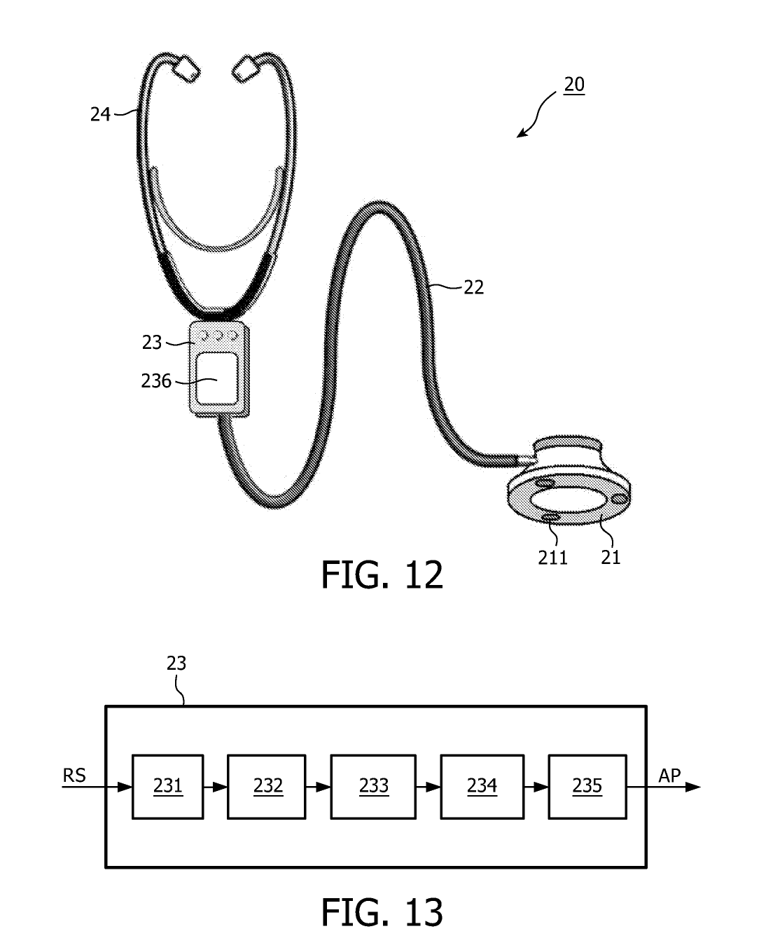

FIG. 12 is a schematic diagram for illustrating a stethoscope. The stethoscope 20 comprises a detecting device 21, a processing system 23, and a connector 22 for connecting the detecting device 21 to the processing system 23.

The detecting device 21 comprises one or more PCG sensors 211. In FIG. 12, three PCG sensors 211 are shown for detecting heart sound signals. The detecting device 21 may also comprise one or more ECG sensors, and in FIG. 12, the ECG sensor 212 is not shown. In another embodiment, the detecting device 21 may comprise a plurality of ECG sensors, and each ECG sensor is combined with a PCG sensor for touching on body at a same location to detecting ECG signal and PCG signal synchronously. The signal detecting device 21 can move on a body or sucked on a body. The each combination of ECG sensor and PCG sensor may move on a body or sucked on a body.

The connector 22 is used for connecting the signal detecting device 21 to the processing system 23, so as to transmit the ECG signals and the heart sound signals detected by the ECG sensors from the sound sensors of the signal detecting device 21 to the processing system 23.

The processing system 23 is used to process the ECG signals and the heart sound signals from the signal detecting device 21. The processing system 23 comprises a display 236 or printer (not shown) for displaying or printing the processed result outputting by the processing system 23. The processing system 23 may be connected to an outside printer or display to print or display the processed result outputting by the processing system 23.

The stethoscope 20 further comprises a pair of earphones used by people to listen to the heart sounds detected by the sound sensors 211 of the signal detecting device 21.

FIG. 13 depicts a processing system for processing at least one heart sound signal in accordance with an embodiment of the stethoscope of FIG. 12. The processing system 23 comprises a receiving unit 231 for receiving at least one heart sound signal and at least one ECG signal from the detecting device 21, a segmenting unit 232 for segmenting the at least one heart sound signal into a plurality of segments, an identifying unit 233 for identifying attribute information for each segment, an annotating unit 234 for annotating each segment with corresponding attribute information, and an outputting unit 235 for outputting 15 an annotated Phonocardiogram for the segments.

The annotated PCG is more understandable, so that people can make a diagnosis conveniently and accurately.

(1) The receiving unit 231 is used for receiving the at least one heart sound signal.

The at least one heart sound signal may comprise one heart sound signal, or multiple heart sound signals coming from different heart sound sources. The multiple heart sound signals can be two or more heart sound signals. Each heart sound signal is detected by sound sensor placed on a heart sound source, such as mitral area, tricuspid area, aortic area, pulmonary area.

A heart sound signal may comprise several segments which belong to different signal segment types, for example, S1 segment, S2 segment, S3 segment, S4 segment, murmurs segment. S1 is caused by the closure of mitral and tricuspid valves; S2 occurs during the closure of aortic and pulmonary valves; S3 is due to the fast ventricular filling during early diastole; S4 occurs as the result of atria contractions displacing blood into the distended ventricular; murmurs are most likely to be caused by turbulent blood flow. S1 may further comprise M1 caused by Mitral and T1 caused by tricuspid, and S2 may further comprise A2 caused by Aortic and P2 caused by Pulmonic valves. S3, S4 and murmurs are usually inaudible.

The at least one heart sound signal is raw heart sound signal and shown as RS in FIG. 13.

(2) The Segmenting Unit 232 is Used for Segmenting the at Least One Heart Sound signal into a plurality of segments.

If the at least one sound signal comprises multiple heart sound signals, the segmenting step 12 is used to segment the multiple heart sound signals separately.

The segmenting unit 232 may be used to segment the at least one heart sound signal by the way of filtering the heart sound signal by a band-pass filter for selecting a wave band of the heart sound signal and extracting segments from the wave band, if the average amplitude change rate of a segment is higher than a predefined change rate threshold, wherein the wave band is a predefined frequency range; or filtering the heart sound signal into an envelogram and extracting segments from the envelogram, if the average amplitude of a region around a peak point of the heart sound signal exceeds a predefined amplitude threshold.

(3) The Identifying Unit 233 is Used to Identify Attribute Information for Each segment.

The attribute information comprises the type of each segment, the duration of each segment, the timing of each segment, the amplitude of each segment, and/or the intensity of each segment etc. The type of each segment can be S1, S2, S3, S4, and murmurs.

The identifying unit 233 may be used to identify the attribute information of each segment according to the waveform of each segment, relationships of the segments, or jointing an ECG with the PCG of the heart sound signal, wherein the ECG signal is synchronous with the heart sound signal.

(4) The annotating unit 234 is used for annotating each segment with corresponding attribute information.

The annotating unit 234 is used to annotate each segment with the type of S1, S2, S3, S4, or murmur according to the identified attribute information. The annotating unit 234 is further used to annotate each segment with amplitude, duration, intensity etc. according the identified attribute information.

(5) The Outputting Unit 235 is Used to Output an Annotated PCG for the at Least One Heart Sound Signal.

The outputted Phonocardiogram comprises a plurality of segments, and each segment is annotated with corresponding type, amplitude, duration, intensity, timing etc., so that people can recognize problems of the heart sound signal conveniently and accurately. The annotated PCG is shown as AP in FIG. 13.

The annotated Phonocardiogram is to be displayed in the form of bar-shaped diagram, and the height of a bar indicates the average amplitude of each segment, and the width of a bar indicates the duration of each segment.

The processing system 23 for processing the at least one heart sound signal further comprises a comparing unit and a generating unit (not shown in FIG. 13).

(6) Comparing Unit

The comparing unit is used to compare two annotated PCGs to acquire a comparison result, if the at least one heart sound signal comprises multiple heart sound signals, and the multiple heart sound signals come from different heart sound sources respectively. The comparison result comprises similarities and differences of any two annotated PCGs which are compared with each other.

The annotating unit 234 is further used to annotate the comparison result on any one of the PCGs which are compared with each other to form a comparison PCG.

The outputting unit 235 is further intended to output the comparison PCG.

The comparing unit is used to compare the average amplitude and duration of two annotated PCGs. For example, one annotated PCG is from tricuspid area (denoted as PCG_T in the following) and another annotated PCG is from aortic area (denoted as PCG_A in the following). In PCG_A, S2 has bigger amplitude and longer duration, so S2 of PCG_A is more easily identified, then the annotating unit 234 is intended to annotate “wider & higher on PCG_A” for this S2 segment on the comparison PCG. In some cases, S2 is not detected on PCG_T, but it can be correctly identified on PCG_A, and then the annotating unit 234 is intended to annotate on the comparison PCG “only on PCG_A” for this S2 segment. The comparison PCG can be generated based on PCG_A or PCG_T.

Based on the comparison PCG, two PCGs complement with each other to provide more accurate information than using single-channel PCG. Furthermore, the presence of abnormal heart sounds, e.g. S3, S4 and murmurs, can be determined conveniently based on the comparison PCG.

Some recurrent sounds are detected on PCG_T but not on PCG_A, and the segments of the recurrent sounds are annotated as “only on PCG_T”, which shows that the recurrent sounds are not noise, and the source of the sound is near tricuspid area but far from aortic area. Furthermore, several kinds of murmurs appear between S1 segment and S2 segment, such as systolic ejection murmurs, ventricular outflow obstruction murmurs, systolic regurgitation murmurs, ventricular septal defect murmur. The comparison PCG reflects the ventricular septal defect murmur very well because such murmur sound is easily audible at PCG_T but not distinct at the PCG_A. In this way, a physician can reach fast and accurate conclusion to the heart condition.

(7) Generating Unit

The generating unit is used to generate a heart rate information table for the heart sound signal by extracting heart cycle samples from the heart sound signal, wherein the heart rate information table comprises different heart rate categories, a typical heart cycle PCG for each heart rate category.

The outputting unit 235 is also intended to output the heart rate information table for the heart sound signal.

The heart cycle samples are extracted by jointing an ECG and a PCG of the heart sound signal which is synchronous with the ECG signal.

The generating unit may be intended to generate the heart rate information table by the way of:
receiving an ECG signal, wherein the ECG signal and the heart sound signal are synchronous.
extracting heart cycle samples from the heart sound signal by making use of the periodicity of the appearances of R waves and R-peak as a beat delimiter for both ECG and the PCG of the heart sound signal, wherein the R wave is the steepest wave along the ECG waveform and the R-peak is the peak point of the R wave.
calculating heart rate for each heart cycle sample. For example, if the heart cycle sample is 1 second, then the heart rate corresponding to the heart cycle is 60 beats/minute.
categorizing the heart cycle samples into different heart rate categories, wherein the heart cycle samples in the same heart rate category have the same heart rate.
eliminating noise by adding all heart cycle samples of the same heart rate together to forming a typical heart cycle PCG for the heart rate. For example, to directly add the aligned bit of amplitude values of the heart cycle samples to eliminate noise. The heart cycle samples include S1, S2, S3, S4, murmurs (if there are murmurs) which are recurrent and demonstrate strong similarity between one heart cycle and another. The eliminating will not affect the quality of the heart cycle samples. The noise, on the other hand, is Gaussian-like, and can be counteracted by accumulation operation. The new data sequence generated by adding the heart cycle samples is referred as typical heart cycle, which has higher SNR (Signal-Noise Rate) than the heart cycle samples. And the more heart cycle samples are accumulated, the higher SNR is achieved. For example, if 20 heart cycle samples are summed up for the same heart rate category, the SNR increases approximately 20 dB. It should note that for the same heart rate, the length of heart cycle samples is almost identical. Thus the heart cycle samples can be added up without or with minor truncation/stretching.
forming a heart rate information table, wherein the heart rate information table comprises different heart rate categories, a typical heart cycle PCG for each heart rate category, and an annotated heart cycle PCG for each heart rate category.

When presented with such a heart rate information table, people may easily identify abnormal heart sounds and further learn at which heart rate the heart condition of the patient becomes worse.

It should be noted that the above-mentioned embodiments illustrate rather than limit the invention and that those skilled in the art will be able to design alternative embodiments without departing from the scope of the appended claims. In the claims, any reference signs placed between parentheses shall not be construed as limiting the claim. The word “comprising” does not exclude the presence of elements or steps not listed in a claim or in the description. The word “a” or “an” preceding an element does not exclude the presence of a plurality of such elements. The invention can be implemented by unit of hardware comprising several distinct elements and by unit of a programmed computer. In the system claims enumerating several units, several of these units can be embodied by one and the same item of hardware or software. The usage of the words first, second and third, et cetera, does not indicate any ordering. These words are to be interpreted as names.

1. A method of processing at least one heart sound signal, comprising the steps of:
receiving (11) the at least one heart sound signal,
segmenting (12) the heart sound signal into a plurality of segments,
identifying (13) attribute information for each segment,
annotating (14) each segment with corresponding attribute information, and
outputting (15) an annotated Phonocardiogram for the at least one heart sound signal.

2. A method as claimed in claim 1, wherein the segmenting step (12) is intended to segment the at least one heart sound signal by
filtering the heart sound signal by a band-pass filter for selecting a wave band of the heart sound signal and extracting segments from the wave band, if the average amplitude change rate of a segment is higher than a predefined change rate threshold, wherein the wave band is a predefined frequency range; or
filtering the heart sound signal into an envelogram and extracting segments from the envelogram, if the average amplitude of a region around a peak point of the heart sound signal exceeds a predefined amplitude threshold.

3. A method as claimed in claim 1, wherein the identifying step (13) is intended to identify the attribute information of each segment according to the waveform of each segment, relationships of the segments, or jointing an Electrocardiogram with the Phonocardiogram of the at least one heart sound signal, wherein the signal of the Electrocardiogram is synchronous with the at least one heart sound signal.

4. A method as claimed in claim 1, further comprising a step of comparing two annotated Phonocardiograms to acquire a comparison result, if the at least one heart sound signal comprises multiple heart sound signals, and the multiple heart sound signals come from different heart sound sources respectively,
wherein,
the annotating step (14) is further intended to annotate the comparison result on any one of the Phonocardiograms which are compared with each other to form a comparison Phonocardiogram, and
the outputting step (15) is further intended to output the comparison Phonocardiogram.

5. A method as claimed in claim 1, further comprising a step of generating a heart rate information table for the heart sound signal by extracting heart cycle samples from the heart sound signal, wherein the heart rate information table comprises different heart rate categories, a typical heart cycle Phonocardiogram for each heart rate category, and an annotated heart cycle Phonocardiogram for each heart rate,
wherein,
the outputting step (15) is further intended to output the heart rate information table for the heart sound signal.

6. A method as claimed in claim 5, wherein generating step is intended to extract the heart cycle sample by jointing an Electrocardiogram with the Phonocardiogram of the at least one heart sound signal, wherein the signal of Electrocardiogram is synchronous with the at least one heart sound signal.

7. A method as claimed in claim 6, wherein the generating step is intended to:
calculate heart rate for each heart cycle,
categorize the heart cycle samples into different heart rate categories, wherein the heart cycle samples in the same heart rate category have the same heart rate,
eliminate noise by adding all heart cycle samples of the same heart rate category together to form a typical heart cycle Phonocardiogram for the heart rate category, and
form the heart rate information table.

8. A method as claimed in claim 1, wherein the attribute information comprises the type of each segment, the duration of each segment, the timing of each segment, the amplitude of each segment, and/or the intensity of each segment.

9. A processing system (23) for processing at least one heart sound signal comprising:
a receiving unit (231) for receiving the at least one heart sound signal,
a segmenting unit (232) for segmenting the heart sound signal into a plurality of segments,
an identifying unit (233) for identifying attribute information for each segment,
an annotating unit (234) for annotating each segment with corresponding attribute information, and
an outputting unit (235) for outputting an annotated Phonocardiogram for the at least one heart sound signal.

10. A processing system as claimed in claim 9, wherein the segmenting unit (232) is intended to segment the at least one heart sound signal by
filtering the heart sound signal by a band-pass filter for selecting a wave band of the heart sound signal and extracting segments from the wave band, if the average amplitude change rate of a segment is higher than a predefined change rate threshold, wherein the wave band is a predefined frequency range; or
filtering the heart sound signal into an envelogram and extracting segments from the envelogram, if the average amplitude of a region around a peak point of the heart sound signal exceeds a predefined amplitude threshold.

11. A processing system as claimed in claim 9, wherein the identifying unit (233) is intended to identify the attribute information of each segment according to the waveform of each segment, relationships of the segments, or jointing an Electrocardiogram with the Phonocardiogram of the at least one heart sound signal, wherein the signal of the Electrocardiogram is synchronous with the at least one heart sound signal.

12. A processing system as claimed in claim 9, further comprising a comparing unit for comparing two annotated Phonocardiograms to acquire a comparison result, if the at least one heart sound signal comprises multiple heart sound signals, and the multiple heart sound signals come from different heart sound sources respectively,
wherein,
the annotating unit (234) is further intended to annotate the comparison result on any one of the Phonocardiograms which are compared with each other to form a comparison Phonocardiogram, and
the outputting unit (235) is further intended to output the comparison Phonocardiogram.

13. A processing system as claimed in claim 9, further comprising a generating unit for generating a heart rate information table for the heart sound signal by extracting heart cycle samples from the at least one heart sound signal, wherein the heart rate information table comprises different heart rate categories, a typical heart cycle Phonocardiogram for each heart rate category, and an annotated heart cycle Phonocardiogram for each heart rate, and an annotated heart cycle Phonocardiogram,
wherein,
the outputting unit (235) is further intended to output the heart rate information table for the heart sound signal.

14. A system as claimed in claim 13, wherein generating unit is intended to
extract the heart cycle sample by jointing an Electrocardiogram with an Phonocardiogram of the at least one heart sound signal, wherein the signal of Electrocardiogram is synchronous with the heart sound signal,
calculate heart rate for each heart cycle,
categorize the heart cycle samples into different heart rate categories, wherein the heart cycle samples in the same heart rate category have the same heart rate,
eliminate noise by adding all heart cycle samples of the same heart rate category together to form a typical heart cycle Phonocardiogram for the heart rate category, and
form the heart rate information table.

15. A stethoscope comprising a detecting device (21) and a connector (22) for connecting the processing system (23) as claimed in any claim 9 to the detecting device (21).
